(19) United States
(12) Patent Application Publication
(10) Pub. No.: US 2003/0073636 A1
(43) Pub. Date: Apr. 17, 2003

(54) Method of treating diabetes

(75) Inventor: Fletcher B. Taylor JR., Oklahoma City, OK (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Oklahoma Medical Research Foundation

(21) Appl. No.: 10/246,644

(22) Filed: Sep. 18, 2002

Related U.S. Application Data
(60) Provisional application No. 60/323,529, filed on Sep. 19, 2001.

(51) Int. Cl. A61K 38/17

(52) U.S. Cl. 514/12

(57) ABSTRACT

It has been discovered that the dysfunction of microvascular endothelium associated with microvascular thrombosis associated with diabetes mellitus can be treated by infusion of activated Protein C. As demonstrated by the example, infusion of an insulin-dependent baboon and normal baboons demonstrated that one can normalize the thrombin-antithrombin (TAT), activated protein C/protein C inhibitor (APC/PCI) and protein C( PC).

This application claims priority to U.S. Ser. No. 60/323,529 filed Sep. 19, 2002.

### BACKGROUND OF THE INVENTION

The present invention is a method for treatment of diabetes using activated protein C.

An estimated 16 million people in the United States have diabetes mellitus—a serious, lifelong condition. About one-third of these 16 million people have not yet been diagnosed. Each year, about 800,000 people are diagnosed with diabetes.

Diabetes is a disorder of metabolism. Most of the food we eat is broken down into glucose, the form of sugar in the blood. Glucose is the main source of fuel for the body. After digestion, glucose passes into the bloodstream, where it is used by cells for growth and energy. For glucose to get into cells, insulin must be present. Insulin is a hormone produced by the pancreas, a large gland behind the stomach. The pancreas is supposed to automatically produce the right amount of insulin to move glucose from blood into our cells. In people with diabetes, however, the pancreas either produces little or no insulin, or the cells do not respond appropriately to the insulin that is produced. Glucose builds up in the blood, overflows into the urine, and passes out of the body. Thus, the body loses its main source of fuel even though the blood contains large amounts of glucose.

The three main types of diabetes are Type 1 diabetes, Type 2 diabetes, and Gestational diabetes. Type 1 diabetes is an autoimmune disease in which the immune system attacks the insulin-producing beta cells in the pancreas and destroys them. The pancreas then produces little or no insulin. Someone with type 1 diabetes needs to take insulin daily to live. At present, scientists do not know exactly what causes the body's immune system to attack the beta cells, but they believe that both genetic factors and environmental factors, possibly viruses, are involved. Type 1 diabetes accounts for about 5 to 10 percent of diagnosed diabetes in the United States. Type 1 diabetes develops most often in children and young adults, but the disorder can appear at any age. Symptoms of type 1 diabetes usually develop over a short period, although beta cell destruction can begin years earlier. Symptoms include increased thirst and urination, constant hunger, weight loss, blurred vision, and extreme fatigue. If not diagnosed and treated with insulin, a person can lapse into a life-threatening diabetic coma, also known as diabetic ketoacidosis.

The most common form of diabetes is type 2 diabetes. About 90 to 95 percent of people with diabetes have type 2. This form of diabetes usually develops in adults age 40 and older and is most common in adults over age 55. About 80 percent of people with type 2 diabetes are overweight. Type 2 diabetes is often part of a metabolic syndrome that includes obesity, elevated blood pressure, and high levels of blood lipids. Unfortunately, as more children become overweight, type 2 diabetes is becoming more common in young people. When type 2 diabetes is diagnosed, the pancreas is usually producing enough insulin, but, for unknown reasons, the body cannot use the insulin effectively, a condition called insulin resistance. After several years, insulin production decreases. The result is the same as for type 1 diabetes—glucose builds up in the blood and the body cannot make efficient use of its main source of fuel. The symptoms of type 2 diabetes develop gradually. They are not as sudden in onset as in type 1 diabetes. Some people have no symptoms. Symptoms may include fatigue or nausea, frequent urination, unusual thirst, weight loss, blurred vision, frequent infections, and slow healing of wounds or sores.

Gestational diabetes develops only during pregnancy. Like type 2 diabetes, it occurs more often in African Americans, American Indians, Hispanic Americans, and people with a family history of diabetes. Though it usually disappears after delivery, the mother is at increased risk of getting type 2 diabetes later in life.

People with impaired glucose metabolism, a state between “normal” and “diabetes,” are at risk for developing diabetes, heart attacks, and strokes. There are two forms of impaired glucose metabolism. A person has impaired fasting glucose (IFG) when fasting plasma glucose is 110 to 125 mg/dL. This level is higher than normal but less than the level indicating a diagnosis of diabetes. Approximately 13.4 million people in the United States, or about 7 percent of the population, have IFG. Impaired glucose tolerance (IGT) means that blood glucose during the oral glucose tolerance test is higher than normal but not high enough for a diagnosis of diabetes. IGT is diagnosed when the glucose level is 141 to 199 mg/dL 2 hours after a person is given a drink containing 75 grams of glucose.

Diabetes is widely recognized as one of the leading causes of death and disability in the United States. According to death certificate data, diabetes contributed to the deaths of more than 193,140 people in 1996. Diabetes is associated with long-term complications that affect almost every part of the body. The disease often leads to blindness, heart and blood vessel disease, strokes, kidney failure, amputations, and nerve damage. Uncontrolled diabetes can complicate pregnancy, and birth defects are more common in babies born to women with diabetes.

In 1997, diabetes cost the United States $98 billion. Indirect costs, including disability payments, time lost from work, and premature death, totaled $54 billion; direct medical costs for diabetes care, including hospitalizations, medical care, and treatment supplies, totaled $44 billion.

Before the discovery of insulin in 1921, everyone with type 1 diabetes died within a few years after diagnosis. Although insulin is not considered a cure, its discovery was the first major breakthrough in diabetes treatment. Today, healthy eating, physical activity, and insulin via injection or an insulin pump are the basic therapies for type 1 diabetes. The amount of insulin must be balanced with food intake and daily activities. Blood glucose levels must be closely monitored through frequent blood glucose checking. Healthy eating, physical activity, and blood glucose testing are the basic management tools for type 2 diabetes. In addition, many people with type 2 diabetes require oral medication and insulin to control their blood glucose levels. Even with these treatments, however, complications do arise, particularly with the microcirculation. Decubitus ulcers and amputations as a result of poor circulation are common among those with long standing diabetes.

Eighty percent of patients with diabetes mellitus die a thrombotic death. Carr, J. Diabetes Complications. 15(1):44-54 (2001). Currently there is no treatment for these patients.

It is therefore an object of the present invention to provide a means for treating the microvascular thrombosis associated with diabetes mellitus.

### SUMMARY OF THE INVENTION

It has been discovered that the microvascular thrombosis associated with diabetes mellitus can be treated by infusion of activated Protein C. As demonstrated by the example, infusion of an insulin-dependent baboon and normal baboons demonstrated that one can normalize the thrombin-antithrombin (TAT), activated protein C/protein C inhibitor (APC/PCI) and protein C(PC).

### DETAILED DESCRIPTION OF THE INVENTION

I. Protein C Compositions

Protein C is a naturally occurring Vitamin K dependent protein produced by the liver, which is cleaved by thrombin to yield the more active enzyme, referred to as activated protein C or “APC”. The protein C system represents a major physiological mechanism for anticoagulation. The mechanism of action of the activated form of protein C and the mechanism of activation of the inactive zymogen into the active protease have been clarified in recent years (for review, see J. E. Gardiner and J. H. Griffin, Progress in Hematology, Vol. XIII, pp. 265-278, ed. Elmer B. Brown, Grune and Stratton, Inc., 1983). The coagulation system is best looked at as a chain reaction involving the sequential activation of zymogens into active serine proteases eventually producing the enzyme, thrombin, which through limited proteolysis converts plasma fibrinogen into the insoluble gel, fibrin. Two key events in the coagulation cascade are the conversion of clotting factor X to Xa by clotting factor IXa and the conversion of prothrombin into thrombin by clotting factor Xa. Both of these reactions occur on cell surfaces, most notably the platelet surface, and both reactions require cofactors. The major cofactors, factors V and VIII, in the system circulate as relatively inactive precursors, but when the first few molecules of thrombin are formed, thrombin loops back and activates the cofactors through limited proteolysis. The activated cofactors, Va and VIIIa, accelerate both the conversion of prothrombin into thrombin and also the conversion of factor X to factor Xa by approximately five orders of magnitude. Activated protein C overwhelmingly prefers two plasma protein substrates which it hydrolyzes and irreversibly destroys. These plasma protein substrates are the activated forms of the clotting cofactors, Va and VIIIa. Activated protein C only minimally degrades the inactive precursors, clotting factors V and VIII. Activated protein C in dogs has been shown to sharply increase circulating levels of the major physiological fibrinolytic enzyme, tissue plasminogen activator. Activated protein C has been shown in vitro to enhance the lysis of fibrin in human whole blood. Experiments suggest that this effect is mediated through the interaction with an inhibitor of tissue plasminogen activator.

The activation of protein C involves thrombin, the final serine protease in the coagulation cascade, and an endothelial cell membrane-associated glycoprotein called thrombomodulin. Thrombomodulin forms a tight, stoichiometric complex with thrombin. Thrombomodulin, when complexed with thrombin, totally changes the functional properties of thrombin. Thrombin normally clots fibrinogen, activates platelets, and converts clotting cofactors V and VIII to their activated forms, Va and VIIIa. Finally, thrombin acts on protein C to activate it but only very slowly and very inefficiently. In contrast, thrombin complexed with thrombomodulin does not clot fibrinogen, does not activate platelets, and does not convert clotting factors V and VIII to their activated counterparts. Thrombin in complex with thrombomodulin activates protein C, and the rate constant of protein C activation by thrombomodulin-thrombin is some 20,000 fold higher than the rate constant for thrombin alone.

An important cofactor for activated protein C is protein S, another vitamin K-dependent plasma protein, and protein S substantially increases activated protein C-mediated hydrolysis of factors Va and VIIIa. Activated protein C is an antithrombotic agent with a wider therapeutic index than available anticoagulants, such as heparin and the oral hydroxycoumarin type anticoagulants. Neither protein C nor activated protein C is effective until thrombin is generated at some local site. Activated protein C is virtually ineffective without thrombin, because thrombin is needed to convert clotting factors V to Va and VIII to VIIIa; the activated forms of these two cofactors are the preferred substrate for activated protein C. The protein C zymogen, when infused into patients, will remain inactive until thrombin is generated and complexed with thrombomodulin; for without thrombomodulin-thrombin, the protein C zymogen is not converted into its active counterpart. Thus, protein C or activated protein C is an on-demand anticoagulant of wide clinical utility for use as an alternative to heparin and the hydroxycoumarins. These conventional anticoagulants, in contrast to protein C, maintain a constant anticoagulant state for as long as they are given to the patient, thereby substantially increasing the risk of bleeding complications over that predicted for protein C or activated protein C.

Human protein C is a serine protease zymogen present in blood plasma and synthesized in the liver. For expression of complete biological activity, protein C requires a post-translational modification for which vitamin K is needed. The mature, two-chain, disulfide-linked, protein C zymogen arises from a single-chain precursor by limited proteolysis. This limited proteolysis is believed to include cleavage of a signal peptide of about 33 amino acid residues (residues 1-33, below) during secretion of the nascent polypeptide from the liver, removal of a pro peptide of about 9 amino acid residues (residues 34-42), and removal of amino acid residues 198 and 199 to form the two chains observed in the zymogen. The activation of the zymogen into the active serine protease involves the proteolytic cleavage of an ARG-LEU peptide bond (residues 211 and 212). This latter cleavage releases a dodecapeptide (residues 200-211) constituting the amino-terminus of the larger chain of the two-chain molecule. Protein C is a vitamin K-dependent serine protease produced in an inactive form by the liver. The zymogen circulates in the plasma and is activated by thrombin only at the surface of endothelial cells. The activation occurs when protein C binds to the thrombin complexed to thrombomodulin on the endothelial cell surface. When so activated, the protein C-protein S complex deactivates two of the cofactors of the coagulant pathway, factors Va and VIIIa, thereby inhibiting coagulation.

Protein C is significantly glycosylated; the mature enzyme contains about 23% carbohydrate. Protein C also contains a number of unusual amino acids, including gamma-carboxyglutamic acid and .beta.-hydroxyaspartic acid. .gamma.-carboxyglutamic acid (gla) is produced from glutamic residues with the aid of a hepatic microsomal carboxylase which requires vitamin K as a cofactor. Since prokaryotes usually neither glycosylate, gamma.-carboxylate, nor beta-hydroxylate proteins expressed from recombinant genes Protein C has been sequenced and is routinely produced recombinantly in bacterial or eucaryotic systems. See, for example, U.S. Pat. Nos. 4,775,624 and 4,992,373 Bang, et al. Protein C can also be isolated from plasma. Since it is somewhat species-specific, it is best to use APC of the same species of origin as the patient to be treated. The APC will typically be administered in a pharmaceutically acceptable carrier. In the preferred embodiment, the preparation will be administered intravenously and the carrier should be selected accordingly. Preferred carriers include normal saline, five percent dextrose in water, Lactated Ringer's Solution and other commercially prepared physiological buffer solutions for intravenous infusion. Of course, the selection of the carrier may depend on the subject's needs or condition.

Suitable formulations are described in U.S. Pat. No. 6,159,468. See also U.S. Pat. No. 6,071,514 for combination therapies with activated Protein C in combination with antiplatelet agents. Activated Protein C formulations are approved by the FDA for administration to patients with sepsis and marketed by Eli Lilly under the tradename XIGRIS™.

II. Administration of Activated Protein C

An effective amount of activated protein C is administered to the patient to alleviate the symptoms of the diabetes, and in particular the microvascular thrombosis associated with diabetes mellitus.

Intravenous administration of the preparation usually will involve one or more boluses in conjunction with a continuous infusion of a more dilute solution. The concentration of the dilute solution may be adjusted to accommodate the fluid needs of the patient. The response of the subject will be the primary determinant and this may vary widely depending on the particular needs of the subject and the inflammatory event involved. However, it will be noted that in general, rescue or therapeutic doses will be higher than doses required in prophylactic treatment.

The present invention will be further understood by reference to the following non-limiting examples.

### EXAMPLE 1

### Activation of Protein C following Infusion of Factor XaPCPS is Impaired in the Diabetic Baboon

Dysfunction of the microvascular thrombosis is associated with the diabetes mellitus. It was postulated that this dysfunction included impaired function of the Protein C network.

An insulin-dependent diabetic baboon (HbA1c=13) and two normal baboons were infused with factor Xa (Xa=36 pM/Kg) plus phospholipid vesicles (PCPS=58 nM/kg) and assayed blood samples collected at the time intervals shown below for thrombin-antithrombin (TAT), activated protein C/protein C/protein C inhibitor (APC/PCI) and protein C(PC). These studies were repeated twice on each of these animals.

The results indicate that administration of activated protein C should reduce thrombotic disorders associated with diabetes.

| | NORMAL | | DIABETIC | |
|---|---|---|---|---|
| Times(min) | Range | Average | Range | Average |
| APC/PCI | 199-204 | 202 | 199 | 199 |
| (pM) T-0 | | | | |
| +5 | 693-760 | 726 | 199-212 | 205 |
| +10 | 622-779 | 700 | 232 | 232 |
| +15 | 1046-1277 | 1161 | 383-406 | 394 |
| +30 | 1399-1844 | 1621 | 558-592 | 575 |
| +60 | 1027-1631 | 1329 | 802-897 | 849 |
| +90 | 674-1180 | 927 | 563-750 | 656 |
| +180 | 199-469 | 334 | 199-204 | 201 |
| TAT (Nm) | 1.00-1.00 | 1.00 | 1.90-2.10 | 2.0 |
| T-0 | | | | |
| +5 | 1.73-2.30 | 2.02 | 8.83-12.41 | 10.61 |
| +10 | 1.38-242 | 1.90 | 12.46 | 12.46 |
| +15 | 1.01-1.69 | 1.35 | 12.76-17.20 | 14.98 |
| +30 | 1.00-1.00 | 1.00 | 8.50-13.32 | 10.91 |
| +60 | 1.00-1.00 | 1.00 | 5.34-8.80 | 7.20 |
| +90 | 1.00-1.00 | 1.00 | 2.63-4.00 | 3.32 |
| +180 | 1.00-1.00 | 1.00 | 1.35-1.44 | 1.40 |
| PC Antigen | 95-95 | 95 | 93-102 | 97 |
| (μg/ml) T-0 | | | | |
| +5 | 74-97 | 85 | 88-93 | 90 |
| +10 | 82-91 | 86 | 104 | 104 |
| +15 | 79-96 | 87 | 95-102 | 98 |
| +30 | 73-86 | 79 | 97-117 | 107 |
| +60 | 84-85 | 84 | 97-107 | 102 |
| +90 | 84-85 | 84 | 100-108 | 104 |
| +180 | 83-91 | 87 | 86-93 | 89 |

I claim:

1. A method of treating the dysfunction of microvascular thrombosis associated with diabetes mellitus comprising administering to a patient with diabetes in need thereof an effective amount of activated protein C to alleviate the symptoms associated with the microvascular thrombosis.

2. The method of claim 1 wherein the activated protein C is recombinant human protein C.

3. The method of claim 1 wherein the activated protein C is administered intravascularly.
